(19) United States
(12) Patent Application Publication
(10) Pub. No.: US 2023/0389889 A1
(43) Pub. Date: Dec. 7, 2023

(54) METHOD AND SYSTEM FOR ULTRASONIC NON-INVASIVE TRANSCRANIAL IMAGING EMPLOYING BROADBAND ACOUSTIC METAMATERIAL

(71) Applicant: ZHEJIANG UNIVERSITY, Zhejiang (CN)

(72) Inventors: Yinfei Zheng, Zhejiang (CN); Yuming Yang, Zhejiang (CN); Huilong Duan, Zhejiang (CN)

(21) Appl. No.: 18/248,677

(22) PCT Filed: Sep. 29, 2021
(86) PCT No.: PCT/CN2021/121533

§ 371 Date: Apr. 11, 2023

(30) Foreign Application Priority Data
Oct. 19, 2020 (CN) 202011115204.1

(51) Int. Cl. A61B 8/08; A61B 8/00

(52) U.S. Cl. CPC A61B 8/0808; A61B 8/4281

(57) ABSTRACT

A method and system for ultrasonic non-invasive transcranial imaging employing a broadband acoustic metamaterial is provided. The method includes: obtaining reflected signals corresponding to different acoustic metamaterial parameter combinations and a skull as a whole (S101); determining a to-be-determined acoustic metamaterial parameter combination according to a to-be-determined reflected signal, and a trained three-layer back propagation (BP) neural network (S102); and determining whether the to-be-determined acoustic metamaterial parameter combination is within a threshold space (S103); if yes, preparing an acoustic metamaterial using the to-be-determined acoustic metamaterial parameter combination (S104); and performing ultrasonic non-invasive transcranial imaging on a resolution mold (S105); and if not, performing re-determination (S106). The method and system for ultrasonic non-invasive transcranial imaging enhances the penetration effect of acoustic waves on the skull and realizes ultrasonic non-invasive transcranial imaging.

### CROSS REFERENCE TO RELATED APPLICATION

This application claims priority to the Chinese Patent Application No. 202011115204.1, filed with the China National Intellectual Property Administration (CNIPA) on Oct. 19, 2020, and entitled “METHOD AND SYSTEM FOR ULTRASONIC NON-INVASIVE TRANSCRANIAL IMAGING EMPLOYING BROADBAND ACOUSTIC METAMATERIAL”, which is incorporated herein by reference in its entirety.

### TECHNICAL FIELD

The present disclosure relates to the field of brain imaging, particularly to a method and system for ultrasonic non-invasive transcranial imaging employing a broadband acoustic metamaterial.

### BACKGROUND ART

Because the skull has strong attenuation and distortion effects on ultrasound, it is difficult for the existing ultrasound imaging theory to effectively penetrate various parts of the skull to achieve intracranial tissue and blood flow imaging (hereinafter referred to as transcranial ultrasound imaging). In recent years, world-renowned research institutions such as Harvard University, the Massachusetts Institute of Technology, and the Institute Laue-Langevin in France have successively carried out frontier exploration of transcranial ultrasound imaging: in 2014, Shen et al. proposed the design idea of a new transcranial ultrasound metamaterial. In 2019, Cai et al. proposed a general method for making underwater metamaterials by 3D printing, which provided a new method for the actual fabrication of acoustic metamaterials. In 2015, Errico et al. developed a super-resolution ultrasound brain imaging system for small animals, and in the same year, Arvanitis et al. tried the passive ultrasound imaging method of the brain. In 2019, Alexandre et al. carried out functional ultrasound brain imaging for the first time on primates. However, due to many factors such as the strong distortion effect of the skull on sound waves and the unknown mechanism of acoustic metamaterials, the transcranial ultrasound brain imaging systems are still in the stage of theoretical exploration, and mostly use invasive transcranial imaging.

At present, the foreword exploration of transcranial ultrasound imaging is mostly invasive imaging, which needs to be carried out on the basis of removing or thinning the skull. Based on this, it is necessary to provide a method and system for ultrasonic non-invasive transcranial imaging employing a broadband acoustic metamaterial, so as to enhance the penetration effect of acoustic waves on the skull and realize ultrasonic non-invasive transcranial imaging.

### SUMMARY

An objective of the present disclosure is to provide a method and system for ultrasonic non-invasive transcranial imaging employing a broadband acoustic metamaterial, so as to enhance the penetration effect of acoustic waves on the skull and realize ultrasonic non-invasive transcranial imaging.

In order to achieve the above objective, the present disclosure provides the following technical solutions:

A method for ultrasonic non-invasive transcranial imaging employing a broadband acoustic metamaterial includes the following steps:
obtaining reflected signals corresponding to different acoustic metamaterial parameter combinations and a skull as a whole, where acoustic metamaterial parameters include an average particle size, a doping ratio, a thickness, and a matrix molecular weight;
determining a to-be-determined acoustic metamaterial parameter combination according to a to-be-determined reflected signal and a trained three-layer back propagation (BP) neural network, where the trained three-layer BP neural network takes the reflected signal as an input and takes the acoustic metamaterial parameter combination corresponding to the reflected signal as an output; and
determining whether the to-be-determined acoustic metamaterial parameter combination is within a threshold space;
if yes, preparing an acoustic metamaterial using the to-be-determined acoustic metamaterial parameter combination; and
performing ultrasonic non-invasive transcranial imaging according to the prepared acoustic metamaterial and a resolution mold; and
if not, updating the to-be-determined reflected signal, replacing the to-be-determined reflected signal with the updated to-be-determined reflected signal, and returning to the step of “determining a to-be-determined acoustic metamaterial parameter combination according to a to-be-determined reflected signal and a trained three-layer BP neural network”.

Optionally, the step of obtaining reflected signals corresponding to different acoustic metamaterial parameter combinations and the skull as a whole may specifically include: preparing the acoustic metamaterial corresponding to the different acoustic metamaterial parameter combinations;
taking the prepared acoustic metamaterial and the skull as a to-be-acquired portion; and
obtaining a reflected signal of the to-be-acquired portion according to a probe, where the reflected signal may be a signal with a minimum amplitude in reflected echo signals.

Optionally, the method may further include the following step after the step of obtaining reflected signals corresponding to different acoustic metamaterial parameter combinations and the skull as a whole:
performing normalization processing on reflected signals corresponding to the different acoustic metamaterial parameter combinations.

Optionally, the method may further include the following steps before the step of determining a to-be-determined acoustic metamaterial parameter combination according to a to-be-determined reflected signal and a trained three-layer BP neural network:
constructing the three-layer BP neural network according to the different acoustic metamaterial parameter combinations and reflected signals corresponding to the different acoustic metamaterial parameter combinations; and
training the three-layer BP neural network using the different acoustic metamaterial parameter combinations.

A system for ultrasonic non-invasive transcranial imaging employing a broadband acoustic metamaterial includes:
a reflected signal obtaining module configured to obtain reflected signals corresponding to different acoustic metamaterial parameter combinations and a skull as a whole, where acoustic metamaterial parameters include an average particle size, a doping ratio, a thickness, and a matrix molecular weight;
an acoustic metamaterial parameter combination determination module configured to determine a to-be-determined acoustic metamaterial parameter combination according to a to-be-determined reflected signal and a trained three-layer BP neural network, where the trained three-layer BP neural network takes the reflected signal as an input and takes the acoustic metamaterial parameter combination corresponding to the reflected signal as an output;
a first determination module configured to determine whether the to-be-determined acoustic metamaterial parameter combination is within a threshold space;
an acoustic metamaterial preparation module configured to prepare an acoustic metamaterial using the to-be-determined acoustic metamaterial parameter combination if the to-be-determined acoustic metamaterial parameter combination is within the threshold space;
an ultrasonic non-invasive transcranial imaging module configured to perform ultrasonic non-invasive transcranial imaging according to the prepared acoustic metamaterial and a resolution mold; and
a to-be-determined reflected signal updating module configured to update the to-be-determined reflected signal, replace the to-be-determined reflected signal with the updated to-be-determined reflected signal, and return to the step of “determining a to-be-determined acoustic metamaterial parameter combination according to a to-be-determined reflected signal and a trained three-layer BP neural network” if the to-be-determined acoustic metamaterial parameter combination is not within the threshold space.

Optionally, the reflected signal obtaining module may specifically include:
an acoustic metamaterial preparation unit configured to prepare the acoustic metamaterial corresponding to the different acoustic metamaterial parameter combinations;
a to-be-acquired portion determination unit configured to take the prepared acoustic metamaterial and the skull as a to-be-acquired portion; and
a reflected signal determination unit configured to obtain a reflected signal of the to-be-acquired portion according to a probe, where the reflected signal may be a signal with a minimum amplitude in reflected echo signals.

Optionally, the system may further include:
a normalization processing module configured to perform normalization processing on reflected signals corresponding to the different acoustic metamaterial parameter combinations.

Optionally, the system may further include:
a three-layer BP neural network construction module configured to construct the three-layer BP neural network according to the different acoustic metamaterial parameter combinations and reflected signals corresponding to the different acoustic metamaterial parameter combinations; and
a three-layer BP neural network training module configured to train the three-layer BP neural network using the different acoustic metamaterial parameter combinations.

According to the specific embodiments provided by the present disclosure, the present disclosure discloses the following technical effects:

According to the method and system for ultrasonic non-invasive transcranial imaging employing a broadband acoustic metamaterial provided by the present disclosure, the to-be-determined acoustic metamaterial parameter combination is determined according to the to-be-determined reflected signal and the trained three-layer BP neural network. That is, a mapping relationship between the reflected signal and preparation parameters of the metamaterial (an average particle size, a doping ratio, a thickness, and a matrix molecular weight) is sought through the neural network method, so as to finally prepare the acoustic metamaterial with the minimum reflected signal. The present disclosure uses the acoustic metamaterial to enhance the characteristics of penetrating the skull without removing or thinning the skull for brain imaging research. The method and system for ultrasonic non-invasive transcranial imaging enhances the penetration effect of acoustic waves on the skull and realizes ultrasonic non-invasive transcranial imaging.

### BRIEF DESCRIPTION OF THE DRAWINGS

To describe the technical solutions in the embodiments of the present disclosure or the prior art more clearly, the accompanying drawings required for the embodiments are briefly described below. Apparently, the accompanying drawings in the following description show merely some embodiments of the present disclosure, and those of ordinary skill in the art may still derive other accompanying drawings from these accompanying drawings without creative efforts.

FIG. 1 is a flow diagram of a method for ultrasonic non-invasive transcranial imaging employing a broadband acoustic metamaterial provided by the present disclosure;

FIG. 2 is a workflow diagram of a probe;

FIG. 3 is a schematic diagram of a resolution mold;

FIG. 4 is a schematic diagram of performing ultrasonic non-invasive transcranial imaging according to the resolution mold; and

FIG. 5 is a schematic structural diagram of a system for ultrasonic non-invasive transcranial imaging employing a broadband acoustic metamaterial provided by the present disclosure.

### DETAILED DESCRIPTION OF THE EMBODIMENTS

The technical solutions of the embodiments of the present disclosure are clearly and completely described below with reference to the accompanying drawings. Apparently, the described embodiments are merely a part rather than all of the embodiments of the present disclosure. All other embodiments obtained by those of ordinary skill in the art based on the embodiments of the present disclosure without creative efforts shall fall within the protection scope of the present disclosure.

An objective of the present disclosure is to provide a method and system for ultrasonic non-invasive transcranial imaging employing a broadband acoustic metamaterial, so as to enhance the penetration effect of acoustic waves on the skull and realize ultrasonic non-invasive transcranial imaging.

To make the above-mentioned objective, features, and advantages of the present disclosure clearer and more comprehensible, the present disclosure will be further described in detail below in conjunction with the accompanying drawings and specific embodiments.

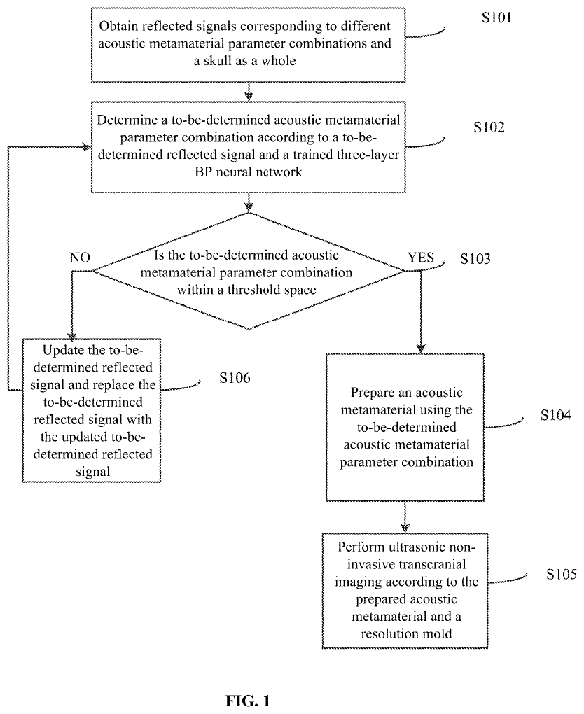

FIG. 1 is a flow diagram of a method for ultrasonic non-invasive transcranial imaging employing a broadband acoustic metamaterial provided by the present disclosure. As shown in FIG. 1, the method for ultrasonic non-invasive transcranial imaging employing a broadband acoustic metamaterial provided by the present disclosure includes the following steps.

S101, Reflected signals corresponding to different acoustic metamaterial parameter combinations and a skull as a whole are obtained. Acoustic metamaterial parameters include an average particle size, a doping ratio, a thickness, and a matrix molecular weight.

S101 specifically includes the following sub-steps.

The acoustic metamaterial corresponding to the different acoustic metamaterial parameter combinations is prepared.

The prepared acoustic metamaterial and the skull are taken as a to-be-acquired portion.

A reflected signal of the to-be-acquired portion is obtained according to a probe. The reflected signal is a signal with a minimum amplitude in reflected echo signals.

An objective of obtaining the reflected signal of the to-be-acquired portion according to the probe is to indirectly obtain the transmitted signal energy of the acoustic metamaterial and the skull as a whole. Because the internal structure of the skull needs to be imaged, the energy to penetrate the skull needs to be very high. That is, the transmitted energy is very large. However, it is not practical to measure the transmitted signal for actual clinical use, and a probe needs to be arranged inside the skull to receive energy. In addition, because energy=transmission+reflection+absorption, the energy absorbed by the same skull remains unchanged, so in the case of the same total energy, smaller reflected energy indicates larger corresponding transmitted energy. Therefore, the overall minimum reflected signal can also be regarded as the maximum transmitted signal, the maximum penetration energy.

A working mode 1 of the probe is used to acquire the reflected signal R of the acoustic metamaterial and the skull as a whole, and the reflected signal is a specific amplitude.

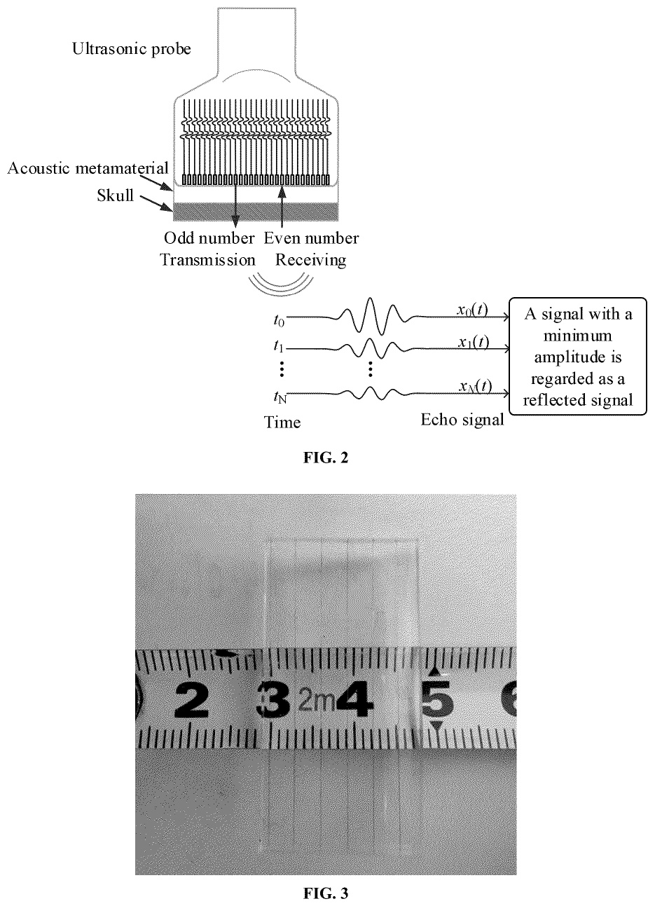

A method for measuring the reflected signal (mode 1 of the probe) is completed by different number of array elements of the probe. The odd number of array elements transmits pulsed ultrasonic waves. After the acoustic wave passes through the material and the upper and lower surfaces of the skull, the reflected echo signal returns. The even number of array elements receives the echoed radio frequency (RF) data after the metamaterial and skull combination. Because the acoustic wave has reflected echo signals after passing through the material and the upper and lower surfaces of the skull, the reflected signal of the material and the skull as a whole has the strongest attenuation in all the reflected echo signals, so its amplitude is the smallest. A method for selecting the reflected signal is as follows: all the echo signals received by the even number of array elements are sorted, and the minimum value obtained by traversal is regarded as the reflected signal of the material and the skull as a whole. A specific workflow is shown in FIG. 2.

Through different acoustic metamaterial parameter combinations (an average particle size A, a doping ratio B, a thickness C, and a matrix molecular weight D), N groups (N>1,000) of metamaterials are prepared, and the reflected signals of the metamaterials and the skull as a whole are acquired in sequence.

The specific combination rules of acoustic metamaterial parameters are as follows: the average particle size A increases from 1 μm to 60 μm in 10 μm strides, namely a [1 μm, 60 μm] threshold range, 10 μm strides, a total of 6 groups. The doping ratio B increases from 1% to 50% in 5% strides, namely a [1%, 50%] threshold range, 5% strides, a total of 10 groups. The thickness C increases from 1 mm to 10 mm in 1 mm stride, namely a [1 mm, 10 mm] threshold range, 1 mm stride, a total of 10 groups. The matrix molecular weight D has two groups of 1,700 and 2,200. There are a total of N=6*10*10*2=1,200 groups of materials. The value of N is also variable according to the adjustment of the threshold and the stride.

After S101, the method further includes the following step.

Normalization processing is performed on reflected signals corresponding to the different acoustic metamaterial parameter combinations. That is, the maximum and minimum values are standardized; that is, the difference between the observed value of a specific reflected signal and the minimum value of the N groups of reflected signals is used as the numerator, and the difference between the maximum value of the N groups of reflected signals and the minimum value of the N groups of reflected signals is used as the denominator. The value after normalization processing can be obtained by dividing the two. After the acquired reflected signal is processed by deviation standardization, all the value ranges exist in [0, 1], which eliminates the size difference between the data and makes all the data fall within the sensitive area of the function. A formula of the normalization processing is as follows:

$${X}^{*} = \frac{X - {X}_{min}}{{X}_{max} - {X}_{min}} \begin{matrix} \\ . {X}^{*}\end{matrix}$$

is the normalized reflection data, X is the observed value of a specific reflected signal, Xₘᵢₙ is the minimum value in the reflected signals, and Xₘₐₓ is the maximum value in the reflected signals.

S102, A to-be-determined acoustic metamaterial parameter combination is determined according to a to-be-determined reflected signal and a trained three-layer BP neural network. The trained three-layer BP neural network takes the reflected signal as an input and the acoustic metamaterial parameter combination corresponding to the reflected signal as an output. The number of hidden layer nodes is determined by the following formula, m=\sqrt{n+1}+σ. m is the number of hidden layer nodes, n is the number of input layer nodes, l is the number of output layer nodes, and σ is a constant between 1 and 10. The node distribution of the three-layer neural network is: 1−m−4.

Before S102, the method further includes the following steps.

The three-layer BP neural network is constructed according to the different acoustic metamaterial parameter combinations and reflected signals corresponding to the different acoustic metamaterial parameter combinations.

The three-layer BP neural network is trained using the different acoustic metamaterial parameter combinations.

A specific training process is as follows:

An Adam optimization algorithm is used, a sigmoid activation function is used, a learning rate is 0.01, an error accuracy is 0.008, and a loss function is mean square error (MSE):

$$MSE = \frac{1}{N} {\sum }_{i = 1}^{N} {( {Y}_{i} - {y}_{i} )}^{2}$$

(where Yᵢ is the actual output of the model, yᵢ is the output predicted by the model, and N is the number of samples). When the loss function is less than the error accuracy, the training ends, and the training model is exited.

If overfitting occurs, the dropout regularization method is used to deal with the model. The trained neural network is tested on the test set, and the model performance evaluation indicators are: MSE and mean absolute error (MAE),

$$MAE = \frac{1}{N} {\sum }_{i = 1}^{N} \vert {Y}_{i} - {y}_{i} \vert .$$

When both parameters are ideal, the model test is completed.

S103, Whether the to-be-determined acoustic metamaterial parameter combination is within a threshold space is determined.

S104, If the to-be-determined acoustic metamaterial parameter combination is within the threshold space, an acoustic metamaterial is prepared using the to-be-determined acoustic metamaterial parameter combination. The resolution mold preparation is conducted by wrapping fine metal wires with polydimethylsiloxane (PDMS) to make five molds with metal wire spacings ranging from 1 mm to 5 mm. The physical picture of the mold with a 3 mm metal wire spacing is shown in FIG. 3. The length, width and thickness of the resolution mold are 50 mm*20 mm*2 mm.

S105, Ultrasonic non-invasive transcranial imaging is performed according to the prepared acoustic metamaterial and a resolution mold.

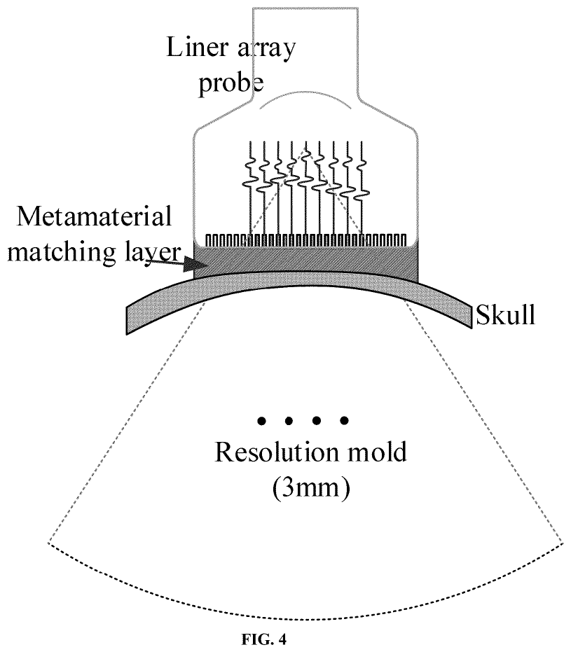

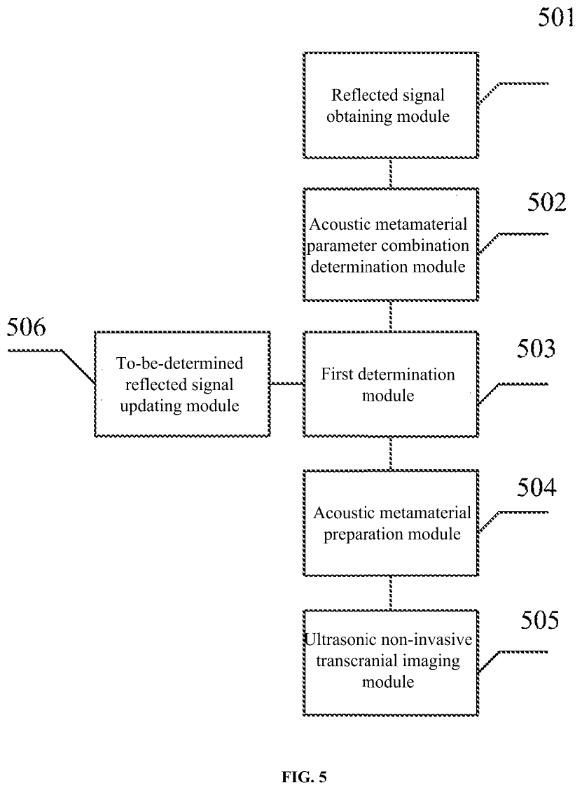

The present disclosure is composed of four parts: a transmitting/receiving probe, an acoustic metamaterial, a skull, and a resolution mold (see FIG. 4 for the device diagram). In working mode 1, the probe is connected to the acoustic metamaterial and the skull, and the reflected signal of the two as unity is measured. In working mode 2, the probe is connected to the acoustic metamaterial, the skull, and the resolution mold. Reflected signal measurement and resolution mold imaging are performed in a water tank filled with degassed distilled water. When the reflected signal is measured, the human skull is placed in the water tank, the ultrasonic probe is placed above the bone fragment through the metamaterial, and the reflected signal of the metamaterial and the skull as a whole is acquired by the probe in the mode 1. When the resolution mold is imaged, the human skull is placed in the water tank, and the resolution mold is parallel to the bone fragment of the skull, placed inside the skull, and moved below the bone fragment about 2 cm away from the foramen magnum. The ultrasonic probe is placed above the bone fragment through the metamaterial, and the resolution mold in the skull is imaged by the probe in mode 2 using the ultrafast compounded plane-wave imaging method.

S106, If the to-be-determined acoustic metamaterial parameter combination is not within the threshold space, the to-be-determined reflected signal is updated, the to-be-determined reflected signal is replaced with the updated to-be-determined reflected signal, and the method returns to S102.

FIG. 5 is a schematic structural diagram of a system for ultrasonic non-invasive transcranial imaging employing a broadband acoustic metamaterial provided by the present disclosure. As shown in FIG. 5, the system for ultrasonic non-invasive transcranial imaging employing a broadband acoustic metamaterial provided by the present disclosure includes a reflected signal obtaining module 501, an acoustic metamaterial parameter combination determination module 502, a first determination module 503, an acoustic metamaterial preparation module 504, an ultrasonic non-invasive transcranial imaging module 505, and a to-be-determined reflected signal updating module 506.

The reflected signal obtaining module 501 is configured to obtain reflected signals corresponding to different acoustic metamaterial parameter combinations and the skull as a whole. Acoustic metamaterial parameters include an average particle size, a doping ratio, a thickness, and a matrix molecular weight.

The acoustic metamaterial parameter combination determination module 502 is configured to determine a to-be-determined acoustic metamaterial parameter combination according to a to-be-determined reflected signal and a trained three-layer BP neural network. The trained three-layer BP neural network takes the reflected signal as an input and the acoustic metamaterial parameter combination corresponding to the reflected signal as an output.

The first determination module 503 is configured to determine whether the to-be-determined acoustic metamaterial parameter combination is within a threshold space.

The acoustic metamaterial preparation module 504 is configured to prepare an acoustic metamaterial using the to-be-determined acoustic metamaterial parameter combination if the to-be-determined acoustic metamaterial parameter combination is within the threshold space.

The ultrasonic non-invasive transcranial imaging module 505 is configured to perform ultrasonic non-invasive transcranial imaging according to the prepared acoustic metamaterial and a resolution mold.

The to-be-determined reflected signal updating module 506 is configured to update the to-be-determined reflected signal, replace the to-be-determined reflected signal with the updated to-be-determined reflected signal, and return to the step of determining a to-be-determined acoustic metamaterial parameter combination according to a to-be-determined reflected signal and a trained three-layer BP neural network if the to-be-determined acoustic metamaterial parameter combination is not within the threshold space.

The reflected signal obtaining module 501 specifically includes an acoustic metamaterial preparation unit, a to-be-acquired portion determination unit, and a reflected signal determination unit.

The acoustic metamaterial preparation unit is configured to prepare the acoustic metamaterial corresponding to the different acoustic metamaterial parameter combinations.

The to-be-acquired portion determination unit is configured to take the prepared acoustic metamaterial and the skull as a to-be-acquired portion.

The reflected signal determination unit is configured to obtain a reflected signal of the to-be-acquired portion according to a probe. The reflected signal is a signal with a minimum amplitude in reflected echo signals.

The system for ultrasonic non-invasive transcranial imaging employing a broadband acoustic metamaterial provided by the present disclosure further includes a normalization processing module.

The normalization processing module is configured to perform normalization processing on reflected signals corresponding to the different acoustic metamaterial parameter combinations.

The system for ultrasonic non-invasive transcranial imaging employing a broadband acoustic metamaterial provided by the present disclosure further includes: a three-layer BP neural network construction module and a three-layer BP neural network training module.

The three-layer BP neural network construction module is configured to construct the three-layer BP neural network according to the different acoustic metamaterial parameter combinations and reflected signals corresponding to the different acoustic metamaterial parameter combinations.

The three-layer BP neural network training module is configured to train the three-layer BP neural network using the different acoustic metamaterial parameter combinations.

Each embodiment of the present specification is described progressively, each embodiment focuses on the difference from other embodiments, and the same and similar parts between the embodiments may refer to each other. Since the system disclosed in an embodiment corresponds to the method disclosed in another embodiment, the description is relatively simple, and reference can be made to the method description.

Specific examples are used herein to explain the principles and embodiments of the present disclosure. The preceding description of the embodiments is merely intended to help understand the method of the present disclosure and its core ideas; besides, various modifications may be made by those of ordinary skill in the art to specific embodiments and the scope of application in accordance with the ideas of the present disclosure. In conclusion, the content of the present specification shall not be construed as limitations to the present disclosure.

What is claimed is:

1. A method for ultrasonic non-invasive transcranial imaging employing a broadband acoustic metamaterial, comprising the following steps:
obtaining reflected signals corresponding to different acoustic metamaterial parameter combinations and a skull as a whole, wherein acoustic metamaterial parameters comprise an average particle size, a doping ratio, a thickness, and a matrix molecular weight;
determining a to-be-determined acoustic metamaterial parameter combination according to a to-be-determined reflected signal and a trained three-layer back propagation (BP) neural network, wherein the trained three-layer BP neural network takes the reflected signal as an input and takes the acoustic metamaterial parameter combination corresponding to the reflected signal as an output; and
determining whether the to-be-determined acoustic metamaterial parameter combination is within a threshold space;
if yes, preparing an acoustic metamaterial using the to-be-determined acoustic metamaterial parameter combination; and
performing ultrasonic non-invasive transcranial imaging according to the prepared acoustic metamaterial and a resolution mold; and
if not, updating the to-be-determined reflected signal, replacing the to-be-determined reflected signal with the updated to-be-determined reflected signal, and returning to the step of “determining a to-be-determined acoustic metamaterial parameter combination according to a to-be-determined reflected signal and a trained three-layer BP neural network”.

2. The method for ultrasonic non-invasive transcranial imaging employing a broadband acoustic metamaterial according to claim 1, wherein the step of obtaining reflected signals corresponding to different acoustic metamaterial parameter combinations and the skull as a whole specifically comprises:
preparing the acoustic metamaterial corresponding to the different acoustic metamaterial parameter combinations;
taking the prepared acoustic metamaterial and the skull as a to-be-acquired portion; and
obtaining a reflected signal of the to-be-acquired portion according to a probe, wherein the reflected signal is a signal with a minimum amplitude in reflected echo signals.

3. The method for ultrasonic non-invasive transcranial imaging employing a broadband acoustic metamaterial according to claim 1, further comprising the following step after the step of obtaining reflected signals corresponding to different acoustic metamaterial parameter combinations and the skull as a whole:
performing normalization processing on reflected signals corresponding to the different acoustic metamaterial parameter combinations.

4. The method for ultrasonic non-invasive transcranial imaging employing a broadband acoustic metamaterial according to claim 1, further comprising the following steps before the step of determining a to-be-determined acoustic metamaterial parameter combination according to a to-be-determined reflected signal and a trained three-layer BP neural network:
constructing the three-layer BP neural network according to the different acoustic metamaterial parameter combinations and reflected signals corresponding to the different acoustic metamaterial parameter combinations; and
training the three-layer BP neural network using the different acoustic metamaterial parameter combinations.

5. A system for ultrasonic non-invasive transcranial imaging employing a broadband acoustic metamaterial, comprising:
a reflected signal obtaining module configured to obtain reflected signals corresponding to different acoustic metamaterial parameter combinations and a skull as a whole, wherein acoustic metamaterial parameters comprise an average particle size, a doping ratio, a thickness, and a matrix molecular weight;
an acoustic metamaterial parameter combination determination module configured to determine a to-be-determined acoustic metamaterial parameter combination according to a to-be-determined reflected signal and a trained three-layer BP neural network, wherein the trained three-layer BP neural network takes the reflected signal as an input and takes the acoustic metamaterial parameter combination corresponding to the reflected signal as an output;
a first determination module configured to determine whether the to-be-determined acoustic metamaterial parameter combination is within a threshold space;
an acoustic metamaterial preparation module configured to prepare an acoustic metamaterial using the to-be-determined acoustic metamaterial parameter combination if the to-be-determined acoustic metamaterial parameter combination is within the threshold space;
an ultrasonic non-invasive transcranial imaging module configured to perform ultrasonic non-invasive transcranial imaging according to the prepared acoustic metamaterial and a resolution mold; and
a to-be-determined reflected signal updating module configured to update the to-be-determined reflected signal, replace the to-be-determined reflected signal with the updated to-be-determined reflected signal, and return to the step of “determining a to-be-determined acoustic metamaterial parameter combination according to a to-be-determined reflected signal and a trained three-layer BP neural network” if the to-be-determined acoustic metamaterial parameter combination is not within the threshold space.

6. The system for ultrasonic non-invasive transcranial imaging employing a broadband acoustic metamaterial according to claim 5, wherein the reflected signal obtaining module specifically comprises:
an acoustic metamaterial preparation unit configured to prepare the acoustic metamaterial corresponding to the different acoustic metamaterial parameter combinations;
a to-be-acquired portion determination unit configured to take the prepared acoustic metamaterial and the skull as a to-be-acquired portion; and
a reflected signal determination unit configured to obtain a reflected signal of the to-be-acquired portion according to a probe, wherein the reflected signal is a signal with a minimum amplitude in reflected echo signals.

7. The system for ultrasonic non-invasive transcranial imaging employing a broadband acoustic metamaterial according to claim 5, further comprising:
a normalization processing module configured to perform normalization processing on reflected signals corresponding to the different acoustic metamaterial parameter combinations.

8. The system for ultrasonic non-invasive transcranial imaging employing a broadband acoustic metamaterial according to claim 5, further comprising:
a three-layer BP neural network construction module configured to construct the three-layer BP neural network according to the different acoustic metamaterial parameter combinations and reflected signals corresponding to the different acoustic metamaterial parameter combinations; and
a three-layer BP neural network training module configured to train the three-layer BP neural network using the different acoustic metamaterial parameter combinations.
